(19) United States
(12) Patent Application Publication
(10) Pub. No.: US 2021/0393141 A1
(43) Pub. Date: Dec. 23, 2021

(54) METHOD AND DEVICE FOR MEASURING HUMAN BODY TEMPERATURE

(71) Applicant: ELO TOUCH SOLUTIONS, INC., Milpitas, CA (US)

(72) Inventors: Haolai ZHOU, Shanghai (CN); Yin SUN, Shanghai (CN); Hua SHAO, Shanghai (CN)

(21) Appl. No.: 17/346,425

(22) Filed: Jun. 14, 2021

(30) Foreign Application Priority Data
Jun. 22, 2020 (CN) 202010575333.2

(51) Int. Cl. A61B 5/01; A61B 5/00; A61B 5/1171

(52) U.S. Cl. CPC A61B 5/015; A61B 5/0064; A61B 5/1176; A61B 2562/0238; A61B 2576/02; A61B 2562/0271; A61B 5/742

(57) ABSTRACT

The present disclosure relates to a method for measuring human body temperature, which comprises: acquiring a distance from an object in front of a temperature measuring device to the temperature measuring device and a visible light image in front of the temperature measuring device; adjusting an orientation of the temperature measuring device according to the visible light image in response to the distance being less than a threshold, so that a specific part of the person is in the measurement area of the temperature measuring device; and measuring a temperature of the specific part of the person in response to the specific part of the person being in the measurement area of the temperature measuring device. The present disclosure further relates to a device for measuring human body temperature.

### CROSS-REFERENCE TO RELATED APPLICATION

This application claims priority to Chinese Patent Application No. 202010575333.2, filed on Jun. 22, 2020, which is hereby incorporated by reference in its entirety.

### TECHNICAL FIELD

The present disclosure relates to methods and devices for measuring human body temperature.

### BACKGROUND

An existing device for measuring human body temperature requires a person to stand in a fixed area in front of the device, and a supervisor adjusts the orientation of the device so that the device is aimed at a specific part of the person, such as the forehead, and then the device measures a temperature of the person.

### SUMMARY

One of the objects of the present disclosure is to provide methods and devices for measuring human body temperature.

A first aspect of this disclosure is to provide a method for measuring human body temperature, which comprises: acquiring a distance from an object in front of a temperature measuring device to the temperature measuring device; acquiring a visible light image in front of the temperature measuring device in response to the distance being less than a threshold; adjusting an orientation of the temperature measuring device according to the visible light image in response to at least part of a person being in the visible light image, so that a specific part of the person is in a measurement area of the temperature measuring device; and measuring a temperature of the specific part of the person in response to the specific part of the person being in the measurement area of the temperature measuring device.

A second aspect of this disclosure is to provide a method for measuring human body temperature, which comprises: acquiring a visible light image in front of the temperature measuring device; acquiring a thermal radiation image in front of the temperature measuring device in response to not any part of a person being in the visible light image; adjusting the orientation of the temperature measuring device according to the thermal radiation image in response to at least part of a person being in the thermal radiation image, so that a specific part of the person is in a measurement area of the temperature measuring device; and measuring a temperature of the specific part of the person in response to the specific part of the person being in the measurement area of the temperature measuring device.

A third aspect of this disclosure is to provide a device for measuring human body temperature, which comprises a processor, a visible light camera and an infrared thermal imager adjacent to the visible light camera, wherein the visible light camera is configured to capture a visible light image in its field of view, and the field of view of the visible light camera includes at least part of the field of view of the infrared thermal imager; and the processor is configured to: control the infrared thermal imager to capture a first thermal radiation image in its field of view in response to not any part of a person being in the visible light image; adjust the orientation of the infrared thermal imager according to the first thermal radiation image in response to at least part of a person being in the first thermal radiation image, so that a specific part of the person is in a measurement area of the infrared thermal imager; and control the infrared thermal imager to capture a second thermal radiation image in its field of view in response to the specific part of the person being in the measurement area of the infrared thermal imager, and measure a temperature of the specific part of the person according to the second thermal radiation image.

A fourth aspect of this disclosure is to provide a device for measuring human body temperature, which comprises a sensing module, an adjusting module, a temperature measuring module, and a control module, wherein the sensing module is configured to sense a position of a person relative to the temperature measuring module in a first direction; the adjusting module is configured to change a position and/or an orientation of the temperature measuring module so as to at least adjust the position of the person relative to the temperature measuring module in a second direction; the temperature measuring module is configured to measure a temperature of an object in a measurement area of the temperature measuring module; and the control module is configured to control the adjusting module to adjust the position of the person relative to the temperature measuring module in the second direction to be in the measurement area, in response to the position of the person relative to the temperature measuring module in the first direction being in the measurement area.

A fifth aspect of this disclosure is to provide a method for measuring human body temperature, which comprises: acquiring a distance from an object in front of a temperature measuring device to the temperature measuring device and a visible light image in front of the temperature measuring device; adjusting the orientation of the temperature measuring device according to the visible light image in response to the distance being less than a threshold, so that a specific part of a person is in the measurement area of the temperature measuring device; and measuring a temperature of the specific part of the person in response to the specific part of the person being in the measurement area of the temperature measuring device.

A sixth aspect of this disclosure is to provide a device for measuring human body temperature, which comprises: one or more processors; and one or more memories configured to store a series of computer executable instructions, wherein the series of computer executable instructions, when executed by the one or more processors, cause the one or more processors to perform the method described above.

A seventh aspect of this disclosure is to provide a non-transitory computer readable storage medium having a series of computer executable instructions stored thereon that, when executed by one or more computing devices, causing the one or more computing devices to perform the method described above.

Further features of the present disclosure and advantages thereof will become apparent from the following detailed description of exemplary embodiments with reference to the attached drawings.

### BRIEF DESCRIPTION OF THE DRAWINGS

The accompanying drawings, which constitute a part of the specification, illustrate embodiments of the present disclosure and, together with the description, serve to explain the principles of the present disclosure.

The present disclosure will be better understood according the following detailed description with reference of the accompanying drawings.

FIG. 1 is a schematic diagram of a device for measuring human body temperature according to an embodiment of the present disclosure.

FIG. 2 is a schematic diagram of the horizontal field of view of the visible light camera and the infrared thermometer in the device of FIG. 1.

FIG. 3 is a schematic diagram of the image of the visible light camera and the measurement area of the infrared thermometer in the device of FIG. 1.

FIGS. 4A and 4B are right views of motion part of the device of FIG. 1 when the motion part rotates downward and upward by a certain angle with respect to the orientation shown in FIG. 1, respectively.

FIGS. 5A and 5B are schematic diagrams of a device for measuring human body temperature according to another embodiment of the present disclosure.

FIGS. 6A and 6B are schematic diagrams of operating states of the device shown in FIGS. 5A and 5B.

FIG. 7 is a schematic diagram of the use of the device of FIGS. 5A and 5B.

FIGS. 8A and 8B are schematic diagrams of the arrangement location of a device for measuring human body temperature according to embodiments of the present disclosure.

FIGS. 9A and 9B are schematic diagrams of the step of adjusting the angle of the orientation of the infrared thermometer in the method for measuring human body temperature according to an embodiment of the present disclosure.

FIG. 10 is a schematic block diagram of a device for measuring human body temperature according to an embodiment of the present disclosure.

FIG. 11 is a schematic block diagram of a device for measuring human body temperature according to an embodiment of the present disclosure.

FIG. 12 is a schematic flowchart of a method for measuring human body temperature according to an embodiment of the present disclosure.

FIG. 13 is a schematic flowchart of a method for measuring human body temperature according to an embodiment of the present disclosure.

Note that, in the embodiments described below, in some cases the same parts or parts having similar functions are denoted by the same reference numerals in different drawings, and description of such parts is not repeated. In some cases, similar reference numerals and letters are used to refer to similar items, and thus once an item is defined in one figure, it need not be further discussed for following figures.

### DETAILED DESCRIPTION

The present disclosure will be described with reference to the accompanying drawings, which show a number of example embodiments thereof. It should be understood, however, that the present disclosure can be embodied in many different ways, and is not limited to the embodiments described below. Rather, the embodiments described below are intended to make the disclosure of the present disclosure more complete and fully convey the scope of the present disclosure to those skilled in the art. It should also be understood that the embodiments disclosed herein can be combined in any way to provide many additional embodiments.

It should be understood that the terminology used herein is for the purpose of describing particular embodiments, but is not intended to limit the present disclosure. All terms (including technical terms and scientific terms) used herein have meanings commonly understood by those skilled in the art unless otherwise defined. For the sake of brevity and/or clarity, well-known functions or structures may be not described in detail.

The term “A or B” used through the specification refers to “A and B” and “A or B” rather than meaning that A and B are exclusive, unless otherwise specified.

The term “exemplary”, as used herein, means “serving as an example, instance, or illustration”, rather than as a “model” that would be exactly duplicated. Any implementation described herein as exemplary is not necessarily to be construed as preferred or advantageous over other implementations. Furthermore, there is no intention to be bound by any expressed or implied theory presented in the preceding technical field, background, summary or detailed description.

In addition, certain terminology, such as the terms “first”, “second” and the like, may also be used in the following description for the purpose of reference only, and thus are not intended to be limiting. For example, the terms “first”, “second” and other such numerical terms referring to structures or elements do not imply a sequence or order unless clearly indicated by the context.

Further, it should be noted that, the terms “comprise”, “include”, “have” and any other variants, as used herein, specify the presence of stated features, integers, steps, operations, elements, and/or components, but do not preclude the presence or addition of one or more other features, integers, steps, operations, elements, components, and/or groups thereof.

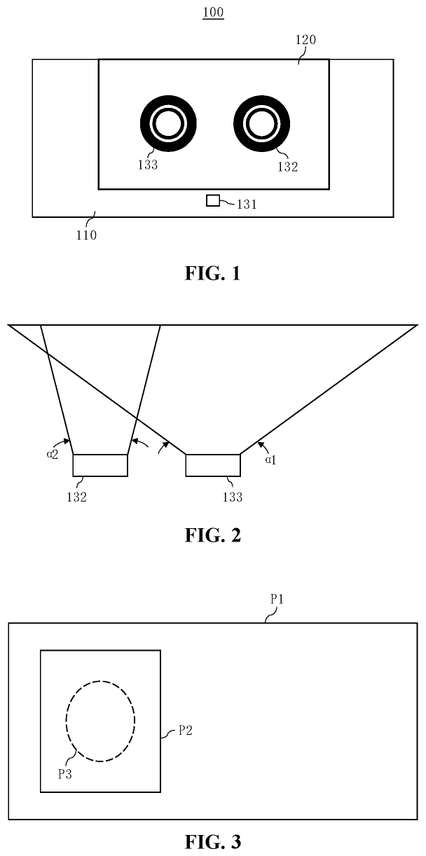

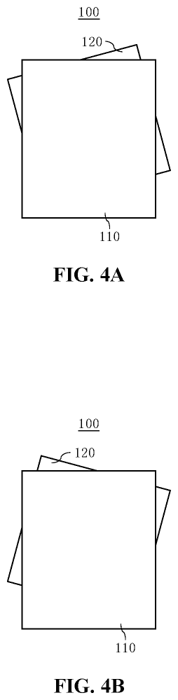

FIG. 1 is a schematic diagram of a device 100 for measuring human body temperature according to an embodiment of the present disclosure. The device 100 includes a base 110 and a motion part 120 that is rotatable relative to the base 110. FIG. 1 is a front view of the device 100 in which the motion part 120 is in its initial position, and FIGS. 4A and 4B are right views of the device 100 in which the motion part 120 is not in its initial position. The motion part 120 shown in FIG. 4A rotates downward by a certain angle with respect to the orientation of its initial position, and the motion part 120 shown in FIG. 4B rotates upward by a certain angle with respect to the orientation of its initial position. In this embodiment, the motion part 120 rotates relative to the base 110 only in the longitudinal direction of the device 100, that is, rotates upward or downward. It will be appreciated that, in another embodiment, the motion part 120 may also rotate relative to the base 110 in the lateral direction of the device 100. In yet another embodiment, the motion part 120 may be movable relative to the base 110 in the longitudinal and/or lateral direction of the device 100. In yet another embodiment, the motion part 120 may be rotatable and movable relative to the base 110.

The device 100 further includes an infrared thermometer 132 and a visible light camera 133 disposed on the motion part 120 adjacent to each other. In the embodiment shown in FIG. 1, the infrared thermometer 132 and the visible light camera 133 are positioned to have the same longitudinal position but different lateral positions. The visible light camera 133 is configured to capture visible light images in its field of view (FOV). The infrared thermometer 132 is configured to measure temperature(s) in its measurement area. In an embodiment, the infrared thermometer 132 may be implemented as an infrared thermal imager, which may be configured to capture thermal radiation images in its FOV. In an embodiment, the infrared thermometer 132 may be implemented as an infrared single-point temperature sensor, such as a commonly used forehead thermometer, which may measure a temperature of a single point. FIG. 2 illustrates a lateral FOV and a field angle α1 of the visible light camera 133, and a lateral FOV and a field angle α2 of the infrared thermometer 132. In the case where the infrared thermometer 132 and the visible light camera 133 are adjacent to each other, as shown in FIG. 3, an FOV P1 of the visible light camera 133 having the wider field angle α1 may include at least part of an FOV P2 of the infrared thermometer 132 having the narrower field angle α2. In an embodiment, at least part of the FOV P2 of the infrared thermometer 132 included in the FOV P1 of the visible light camera 133 may be determined as a measurement area P3 of the infrared thermometer 132. In the embodiment shown in FIG. 3, the FOV P1 of the visible light camera 133 includes the entire FOV P2 of the infrared thermometer 132. In this embodiment, the measurement area P3 of the infrared thermometer 132 may be set as a middle portion of the FOV P2 of the infrared thermometer 132. In the case where the device 100 is configured to measure the temperature of the forehead of the human body, the measurement area P3 may be set as an oval area similar to the shape of the head of the human body.

The infrared thermometer 132 and the visible light camera 133 are disposed on the motion part 120 together. As the motion part 120 rotates and/or moves relative to the base 110, the orientations and/or positions of the infrared thermometer 132 and the visible light camera 133 are changed synchronously. The positions of the two FOVs are also changed synchronously, but the relationship between the positions of the two FOVs will not be changed. Therefore, in an embodiment, the orientation and/or position of the infrared thermometer 132 may be adjusted (through adjusting the orientation and/or position of the motion part 120) by using the visible light image captured by the visible light camera 133 which has a larger observation range (i.e., a wider FOV). It will be appreciated that, in another embodiment, the orientation and/or position of the infrared thermometer 132 may be adjusted on the basis of the heat radiation images captured by the infrared thermometer 132 itself.

The infrared thermometer 132 may be set to an initial position, which generally corresponds to the initial position of the motion part 120. The initial position of the infrared thermometer 132 may be determined as follows: when the device 100 is installed for operation, the FOV of the infrared thermometer 132 may just cover a specific part of a person with a specific height and a specific distance from the device 100. The specific height may be determined according to the average value or mode of the height of the measured crowd, for example, it may be predetermined to be 1.65 meters to 1.75 meters. The specific distance may be the distance between the person and the device 100 when the accuracy and reliability of the measured value of the infrared thermometer 132 meet the requirements. The specific part of the person may include at least one of a face, a forehead, a neck, and a wrist, depending on the purpose configured for the device 100. Since the temperature of the forehead is usually easy to be measured, for the sake of simplicity, some descriptions herein describe the specific part of the person as the face or forehead of the person. The initial position of the infrared thermometer 132 is determined jointly by the position and orientation of the base 110 and the position and orientation of the motion part 120 relative to the base 110. The infrared thermometer 132 may return to its initial position after a measurement is completed, or after a predetermined duration of inactivity, which facilitates the device 100 to always quickly capture the specific part of a person for measurement.

It will be appreciated that the device 100 further includes a means that drives the motion part 120 to motion (rotate and/or move), such as a motor. The motor revolves to drive the motion part 120 to rotate by a specific angle and/or move by a specific displacement, so as to drive the infrared thermometer 132 to change its orientation and/or position. The revolution of the motor may also return the motion part 120 to its initial position, so as to return the infrared thermometer 132 back to its initial position. In an embodiment, the device 100 further includes a position switch that matches the initial position of the motion part 120. During the revolution for returning to the initial position, if the position switch is triggered, the motor stops revolving.

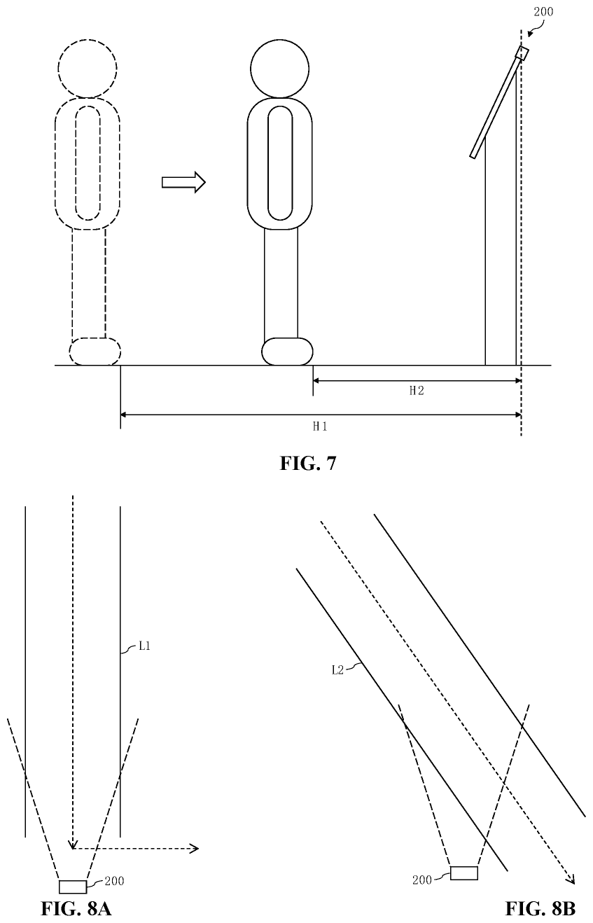

The device 100 further includes a distance sensor 131 for sensing the distance from an object (e.g., a person) in front of the device 100 to the device 100. The measurement distance of the infrared thermometer 132 may be, for example, 5 meters, but the measurement result when the object is within a specific distance range (for example, 0.6 meters to 1 meter) is more accurate and reliable. In an embodiment, with the aid of the distance sensor 131, the temperature of the person may be measured only when the person is within a specific distance range in front of the device 100. In an embodiment, when the distance sensor 131 senses that the distance from the person to the device 100 is less than a threshold (for example, 2 meters), e.g., when the sensed distance is less than the distance H1 shown in FIG. 7, the motion part 120 is triggered to motion so that a specific part of the person could be in the measurement area of the infrared thermometer. At this time, in order to adjust the orientation of the motion part 120, both the infrared thermometer 132 and the visible light camera 133 are usually activated. As such, when a person is far away from the device 100, the infrared thermometer 132 and the visible light camera 133 may not work. As shown in FIG. 7, the person continues to walk forward and gets closer and closer to the device 100 (shown as the device 200 in FIG. 7 which will be described below), and when the distance H2 between the person and the device 100 reaches into a specific distance range (for example, 0.6 m to 1 m), the device 100 has usually been adjusted so that the specific part of the person is in the measurement area of the infrared thermometer 132 already, and the infrared thermometer 132 may perform temperature measuring at this time. It will be appreciated that the device 100 may also perform temperature measuring in response to the specific part of the person being in the measurement area of the infrared thermometer 132, regardless of whether the distance between the person and the device 100 is in a specific distance range. In an embodiment, the device 100 may continuously acquire the distance from the person to the device 100 and continuously measure the temperature of the specific part of the person in response to the specific part of the person being in its measurement area, and record each measured temperature and its corresponding distance. During statistical analysis of these data, the reliability of each temperature value may be determined on the basis of the distance. For example, the temperature value measured within a specific distance range may have a high reliability, and the temperature value measured outside the specific distance range may be used as a reference or backup.

It will be appreciated that in an embodiment, the infrared thermometer 132 and/or the visible light camera 133 may work all the time, that is, capturing image (or measuring temperatures) all the time, and the orientation and/or position of the infrared thermometer 132 may be adjusted according to the image only when the distance sensor 131 senses that the distance between the person and the device 100 is less than the threshold.

In an embodiment, the distance sensor 131 may be oriented so that its orientation is deflected downward than the orientation of the infrared thermometer 132 in the initial position. For example, when the infrared thermometer 132 is in the initial position, the infrared thermometer 132 may generally face forwardly of the device 100, and the distance sensor 131 may face forwardly and downwardly of the device 100. Considering that persons always have body parts near the ground (for example, in the range of 0.3 meters to 0.8 meters from the ground), the distance sensor 131 facing forwardly and downwardly may sense most persons, even if a person whose height is much smaller than the above mentioned average value or mode, such as a child. The distance sensor 131 may be disposed on the base 110 as shown in FIG. 1 to have a relatively fixed orientation and/or position, or may be disposed on the motion part 120 to have its orientation and/or position change in synchronization with the infrared thermometer 132.

It will be appreciated that the device 100 may further include one or more controllers and/or one or more processors to control the device 100 to operate. Although not illustrated, the device 100 may further include some input/output means. In an embodiment, the device 100 may include a speaker to output as sound the value of the temperature measured by the infrared thermometer 132, and/or output as sound that whether the temperature is normal. For example, it may play a sound as “normal body temperature”, “low body temperature”, or “high body temperature”, etc. In an embodiment, the device 100 may include a display to display at least part of the visible light image captured by the visible light camera 133, at least part of the heat radiation image captured by the infrared thermometer 132, the value of the temperature measured by the infrared thermometer 132, an indication of whether the temperature is normal, and/or a user-operable interface. In an embodiment, the device 100 may include a sensor that may sense whether there is an object within a predetermined range in front of it. The sensor may be used to receive an operation from a user. For example, the above display may display a screen to ask the user whether to re-measure. If the user wants to re-measure, the user may place the hand thereof within a predetermined range in front of the sensor (without touching the sensor or the display) staying for a predetermined time period so as to trigger the device 100 to re-measure the body temperature. The sensor allows the user to operate the device 100 without touching. In an example, the sensor may be implemented by a distance sensor. In an embodiment, the display may be a touch screen, which may receive touching operations from the user. In an embodiment, the device 100 may include a printer. The printer may print the measured temperature value after each temperature measurement is completed, or may print the measured temperature value in response to the user's operation (for example, the operation through the sensor described above). The printer may also print a mark indicating that the temperature is normal (for example, in response to the measured temperature being normal), such as an adhesive badge with a specific color. A user (for example, a visitor to an organization) may paste it on clothing to explicitly indicate that his body temperature is normal.

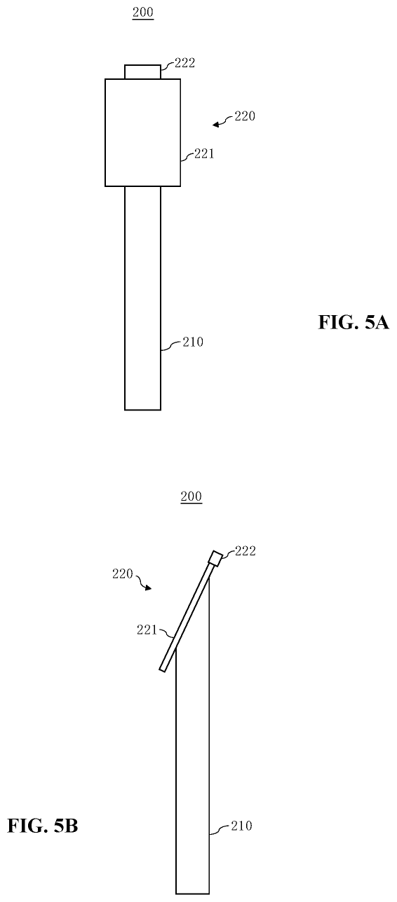

FIGS. 5A and 5B are schematic structural diagrams of a device 200 for measuring human body temperature according to another embodiment of the present disclosure. The device 200 includes a body part 220 and a support part 210 supporting the body part at a predetermined height. The body part 220 includes a first part 221 and a second part 222. The second part 222 may include an entirety or a part of the device 100 as described above. The first part 221 may be a tablet computer, which includes a display and a processor that are configured in a common housing. The second part 222 is mechanically connected to the top of the first part 221. It will be appreciated that in other embodiments, the second part 222 may be connected to any of other portions of the first part 221. The second part 222 may have a controller and/or processor different from the processor of the first part 221, or the second part 222 may have no controller and/or processor and may use the processor of the first part 221 to control the operation of the second part 222. It will be appreciated that there is an interface for data transmission between the first part 221 and the second part 222.

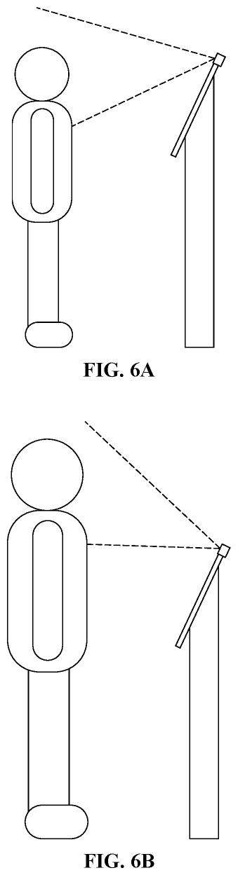

FIGS. 6A and 6B illustrate operating states of the device 200 when measuring temperatures of persons with different heights. As shown in FIG. 6A, when the measured person is short, the orientation of the infrared thermometer of the second part 222 may be deflected downward (for example, in the state shown in FIG. 4A), so that the face of the measured person is in the measurement area of the infrared thermometer. As shown in FIG. 6B, when the measured person is tall, the orientation of the infrared thermometer of the second part 222 may be deflected upward (for example, in the state shown in FIG. 4B), so that the face of the measured person is in the measurement area of the infrared thermometer. In the process of adjusting the orientation of the infrared thermometer, the orientation and position of the support part 210 and the first part 221 of the body part 220 may be maintained unchanged. It will be appreciated that, in other embodiments, the position of the infrared thermometer may be changed, for example, the infrared thermometer is lowered when the measured person is shorter and the infrared thermometer is raised when the measured person is higher, so that the face of the measured person is in the measurement area of the infrared thermometer.

The device 200 with the support part 210 is convenient to be placed near the travel path of the measured crowd. As shown in FIG. 8A, the device 200 may be placed on the travel path L1 of the measured crowd and face the travel direction of the crowd (represented by a dotted line with an arrow). When a person to be measured walks to the device 200 from substantially forward of the device 200, the device 200 automatically measure the human body temperature of the person as shown in FIG. 7. After the measurement is completed, the measured person may leave from a side of the device 200, as shown by the horizontal dotted line in the diagram. As shown in FIG. 8B, the device 200 may be placed beside the travel path L2 of the measured crowd and face the travel direction of the crowd (represented by a dotted line with an arrow). When a person to be measured approaches the device 200 on the path L2, the device 200 may always track the person to be measured by automatically adjusting the orientation of its infrared thermometer, and measuring the human body temperature when the conditions are met. In this case, the measured crowd need not to change the direction of their travel path for the presence of the device 200, so the user experience may be better.

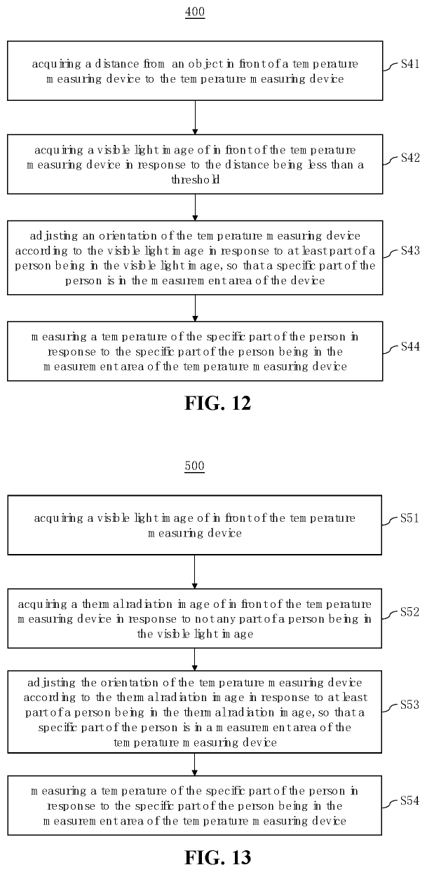

FIG. 12 is a schematic flowchart of a method 400 for measuring human body temperature according to an embodiment of the present disclosure. The method 400 may be executed by the controllers and/or processors in the above-mentioned device 100 or 200 and the device 300 or 600 to be described below. The method 400 includes: acquiring (for example, from the above-mentioned distance sensor) a distance from an object in front of a temperature measuring device (for example, the above-mentioned devices 100, 200) to the temperature measuring device (step S41); in response to the distance being less than a threshold, acquiring (for example, from the above-mentioned visible light camera) a visible light image in front of the temperature measuring device (step S42); in response to at least part of a person being in the visible light image, adjusting an orientation of the temperature measuring device according to the visible light image so that a specific part of the person is in the measurement area of the temperature measuring device (step S43); and in response to the specific part of the person being in the measurement area of the temperature measuring device, measuring (for example, by the above-mentioned infrared thermometer) a temperature of the specific part of the person (step S44).

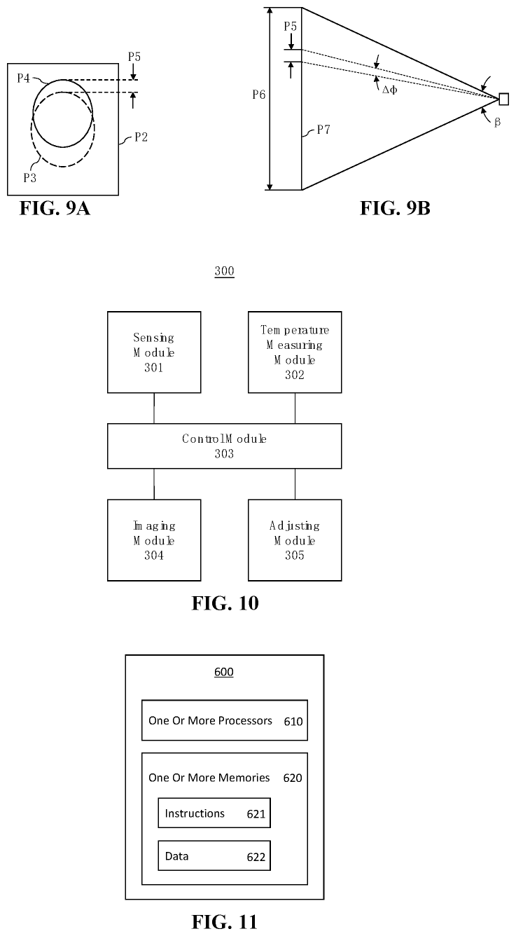

As described above, depending on the purpose for which the temperature measuring device is set, the specific part of the person may include at least one of the face, forehead, neck, and wrist. In the following description, a specific part of a person is sometimes described as the face of a person for simplicity. Known computer vision technology may be used to identify whether at least part of a person is in the visible light image, such as all or a part of a head, an ear, a shoulder, an arm, a torso, a leg, etc. In an example, in response to a shoulder of a person appearing in the visible light image, the orientation of the temperature measuring device is adjusted to deflect upward so that the face of the person is in the measurement area. In an example, in response to an ear of a person being in the visible light image, the orientation of the temperature measuring device is adjusted leftward or rightward so that the face of the person is in the measurement area. A specific example is given below to illustrate how to determine the angle by which the orientation of the temperature measuring device needs to be adjusted. As shown in FIG. 9A, a face P4 of a person appears in the visible light image (which may correspond to the FOV P1 in FIG. 3, which is not shown in FIG. 9A for simplicity). The face P4 is higher than the measurement area P3 and needs to be adjusted into the measurement area P3. The adjustment amount may be determined according to the resolution of the visible light image and the field angle. For example, the vertical field angle β of the visible light camera may be 30 degrees, and the resolution may be 640*480 (that is, the number of pixels P6 in the vertical direction of the visible light image is 480). It may be determined from the visible light image that the interval P5 between the top of the face P4 and the top of the measurement area P3 is 80 pixels, then it may be determined that the angle adjustment amount Δφ may be 30*(80/480) =5 degrees. It will be appreciated that the adjustment amount may also be determined according to a face of a person being in the thermal radiation image (which may correspond to the FOV P2) as described below. In this case, the adjustment amount may be determined according to the resolution of the thermal radiation image and the field angle. Although not shown in the diagram, it will be appreciated by those skilled in the art that the amount of movement that needs to be adjusted for the position of the temperature measuring device may be determined according to a similar method to that in the example.

In order to adjust a specific part of a person into the measurement area in a shorter time, in an embodiment, the method 400 may further include: displaying (for example, through a display) at least part of the visible light image. The displayed image may include the measurement area of the temperature measuring device. The measurement area may be marked with an outline, which allows the user standing in front of the temperature measuring device to intuitively see the position of his face relative to the measurement area, so that the user may adjust the position thereof by himself/herself, for example, by moving forward, backward, left, and right.

In the embodiment shown in FIG. 12, in step S42, a visible light image may be acquired in response to the distance being less than the threshold, that is, the distance sensor senses that there is an object in a predetermined range in front of the temperature measuring device. In an embodiment, in step S42, a visible light image may be acquired in response to the distance being less than the threshold and the distance changing within a first predetermined time, that is, it is sensed that there is a movable object within a predetermined range in front of the temperature measuring device. In an embodiment, in step S42, a visible light image may be acquired in response to the distance being less than the threshold and the distance becoming smaller within the first predetermined time, that is, it is sensed that there is an object within a predetermined range in front of the temperature measuring device and the object is approaching the temperature measuring device.

In an embodiment, the acquired distance is the distance from the object in lower front of the temperature measuring device to the temperature measuring device, for example, it may be acquired by a distance sensor facing face forwardly and downwardly of the device. After the visible light image is acquired in step S42, any part of a person may not be in the visible light image. That is, the distance sensor senses that there is an object in a predetermined range in lower front of the temperature measuring device but the visible light camera does not capture the object. This situation may be caused by the short height of the person near the temperature measuring device (for example, a child) that is lower than the orientation of the visible light camera (for example, the visible light camera may be located in its initial position that matches a height of 1.7 m). In this case, the temperature measuring device may adjust its orientation within its maximum adjustable range while acquiring visible light images in front of the temperature measuring device at its each orientation, until at least part of a person is in the visible light image. That is, scanning is performed by the temperature measuring device in its entire orientation range to capture at least part of a person, so as to adjust the orientation of the temperature measuring device according to the at least part of the person. In this case, the temperature measuring device may scan downwards first so as to capture at least part of a person in a shorter time.

In an embodiment, the temperature of the specific part of the person measured in step S44 may be associated with the distance between the person and the temperature measuring device acquired in step S41. In an embodiment, after step S41, the distance between the person and the temperature measuring device may be continuously acquired, for example, during the execution of steps S42 to S44, and after the execution of step S44. The temperature of the specific part of the person measured in step S44 may be associated with the latest acquired distance between the person and the temperature measuring device. In an embodiment, after adjusting the specific part of the person into the measurement area of the temperature measuring device in step S43, the distance from the person to the temperature measuring device may be continuously acquired (for example, step S41 is periodically performed for times) and the temperature of the specific part of the person may be continuously measured (for example, step S44 is periodically performed for times), and each measured temperature and corresponding distance are recorded. Referring to FIG. 7, in this embodiment, when the distance between the measured person and the temperature measuring device is less than the threshold distance H1 (for example, 2 m), the temperature measuring device starts to adjust its orientation, and at the same time the person continues to walk, either as shown in FIG. 8A, generally towards the temperature measuring device (e.g., facing the device), or as shown in FIG. 8B, slightly biased in the direction towards the temperature measuring device (e.g., facing the device diagonally). When the temperature measuring device is adjusted so that a specific part of the person is within its measurement area, the distance between the person and the temperature measuring device may become 1.6 m, for example between the distances H1 and H2. Then the person continues to walk forward, the temperature measuring device may continue to acquire the distance and temperature value from then on, until the distance between the person and the temperature measuring device is too close to measure, or the person walks out of the measuring range (which is represented by two dashed lines extending outwardly from the device 200 in the diagram) of the temperature measuring device.

In an embodiment, the temperature measuring device may be operable. In response to the distance between the person and the temperature measuring device being greater than an operable distance, the person is prompted to be closer to the temperature measuring device, so that the person may perform contact or non-contact operation on the temperature measuring device.

In an embodiment, if the visible light image acquired in step S42 does not contain any part of a person, the thermal radiation image in front of the temperature measuring device may be acquired (for example, from an infrared thermometer). According to the temperature value of each pixel of the thermal radiation image, it is determined whether at least part of a person is in the thermal radiation image. In response to at least part of the person being in the thermal radiation image, the orientation of the temperature measuring device is adjusted according to the thermal radiation image so that the specific part of the person is in the measurement area of the temperature measuring device. The specific method of recognizing at least part of the person by the thermal radiation image is set forth in the following description for the method 500. In this embodiment, when it is difficult to recognize at least part of a person through a visible light image, a thermal radiation image may be used to further recognize at least part of a person. When neither of the two approaches may identify at least part of a person, the temperature measuring device may adjust its orientation within its maximum adjustable range as described above, that is, scanning is performed by the temperature measuring device within its entire orientation range to capture at least part of a person.

In an embodiment, the method 400 may further include: recognizing an identity of the person and associating the temperature measured by the infrared thermometer with the identity of the person. The identity of a person may be recognized on the basis of visible light images. For example, the identity of the person may be identified on the basis of the face of the person included in the image by using known face recognition technology, or the identity of the person may be identified on the basis of the visual mark contained in the image (such as the name tag worn/carried by the person). In addition, an identification may be read from the external through a reader, so as to recognize the identity of the person based on the identification. In an example, the reader may be an attendance card reader, and the identification may be read from the attendance card. Similarly, the reader may be a fingerprint reader or the like. In an example, the reader may be a QR code reader, and the identification may be read from a QR code. In this case, the reader may be served by the visible light camera, and the method 400 may process the image containing the QR code captured by the visible light camera so as to recognize the identity of the person indicated by the QR code. The temperature measuring device with an identity recognition function may be used as an attendance device for enterprises, schools, kindergartens or other entities.

In an embodiment, the method 400 may further include: displaying the recognized identity, and receiving a contact or non-contact operation from the user which indicates whether the identity is confirmed by the user; and in response to the identity being confirmed, associating the temperature measured by the infrared thermometer with the identity of the person. In an embodiment, the method 400 may further include: outputting the measured temperature value through an output device, such as a display screen, a printer, a speaker, etc.

FIG. 13 is a schematic flowchart of a method 500 for measuring human body temperature according to an embodiment of the present disclosure. The method 500 may be executed by the controllers and/or processors in the above-mentioned device 100 or 200 and the device 300 or 600 to be described below. The method 500 includes: acquiring (for example, from the above-mentioned visible light camera) a visible light image in front of a temperature measuring device (for example, the above-mentioned devices 100, 200) (step S51); in response to not any part of a person being in the visible light image, acquiring (for example, from the above-mentioned infrared thermometer) a thermal radiation image in front of the temperature measuring device (step S52); in response to at least part of a person being in the thermal radiation image, adjusting the orientation of the temperature measuring device according to the thermal radiation image so that a specific part of the person is in the measurement area of the temperature measuring device (step S53); and in response to the specific part of the person being in the measurement area of the temperature measuring device, measuring (for example, by the above infrared thermometer) a temperature of the specific part of the person (step S54).

In this embodiment, if the visible light image acquired in step S51 does not contain any part of a person, then a thermal radiation image is acquired in step S52. According to the temperature value of each pixel of the thermal radiation image, it is determined whether pixels with temperature values greater than a threshold corresponds to at least part of a person. For example, the thermal radiation image may be converted into a binary image according to whether the temperature value of each pixel of the thermal radiation image is greater than a threshold, and the binary image includes one or more regions composed of adjacent pixels with temperature values greater than the threshold. Computer vision technology is used to determine whether at least one region of the one or more regions corresponds to at least part of a person. In response to at least one region of the one or more regions corresponding to at least part of a person, it may be determined that pixels with temperature values greater than the threshold corresponds to at least part of the person. In response to the pixels with temperature values greater than the threshold corresponding to at least part of a person, it may be determined that at least part of a person is in the thermal radiation image, and in step S53, the orientation of the temperature measuring device is adjusted on the basis of the thermal radiation image so that the specific part of the person may be in the measurement area of the temperature measuring device.

The temperature of the exposed part of the human body measured by an infrared thermometer is usually around 32° C. to 34° C., and the temperature of the part covered by clothing is usually around 30° C. Therefore, when the ambient temperature reaches about 30° C., it is not convenient to recognize at least part of the human body from the image through the thermal radiation image. Therefore, in the case of high ambient temperature, such as summer, it is more convenient to use visible light images to recognize at least part of a person. In the case of low ambient temperature, such as winter, people usually wear thicker clothing and use scarves, hats, masks, etc. to partially cover the face, which makes it difficult to recognize at least part of a person through visible light images, especially the face. In this case, the thermal radiation image is used to recognize at least part of a person. Since the temperature difference between the human body and the ambient temperature is large at this time, it is more convenient to use the thermal radiation image to recognize at least part of a person. Therefore, the method 500 may be applied to both hot and cold seasons.

The device for measuring human body temperature working in the method 500 may include a processor, and a visible light camera and an infrared thermal imager adjacent to the visible light camera. The visible light camera is configured to capture visible light images in its FOV, and the FOV of the visible light camera includes at least part of the FOV of the infrared thermal imager. The processor is configured to: in response to not any part of a person being in the visible light image, control the infrared thermal imager to capture a first thermal radiation image in its FOV; in response to at least part of a person being in the first thermal radiation image, adjust the orientation of the infrared thermal imager according to the first thermal radiation image so that a specific part of the person is in the measurement area of the infrared thermal imager; and in response to the specific part of the person being in the measurement area of the infrared thermal imager, control the infrared thermal imager to capture a second thermal radiation image in its FOV, and measure a temperature of the specific part of the person on the basis of the second thermal radiation image. For example, the temperature of the person's forehead may be acquired on the basis of the average value of the temperatures of pixels in the central region of the forehead portion of the person in the second thermal radiation image.

FIG. 10 is a schematic block diagram of a device 300 for measuring human body temperature according to an embodiment of the present disclosure. The device 300 includes a sensing module 301, a temperature measuring module 302, a control module 303, an imaging module 304, and an adjusting module 305. The sensing module 301, the temperature measuring module 302, the imaging module 304, and the adjusting module 305 may be implemented in whole or in part as the above-mentioned distance sensor 131, infrared thermometer 132, visible light camera 133, and motion part 120, respectively. A detailed description of the functions already described is omitted. Both of the above methods 400 and 500 may be executed by the control module 303.

The sensing module 301 is configured to sense a position of a person (which may be a specific part of the person) relative to the temperature measuring module 302 in a first direction (which may be a direction extending forward from the temperature measuring module 302). The adjusting module 305 is configured to change a position and/or orientation of the temperature measuring module 302 so as to at least adjust the position of the person (which may be a specific part of the person) relative to the temperature measuring module 302 in a second direction (which may be a direction of the plane substantially perpendicular to the first direction, e.g., the direction of the imaging plane P7 in FIG. 9B). The temperature measuring module 302 is configured to measure a temperature of an object in its measurement area. The control module 303 is configured to, in response to the position of the person relative to the temperature measuring module 302 in the first direction being in the measurement area, control the adjusting module 305 to adjust the position of the person relative to the temperature measuring module 302 in the second direction to be in the measurement area. The imaging module 304 is configured to capture images of a person. The control module 303 may be further configured to control the adjusting module 305 to adjust the position of the person relative to the temperature measuring module 302 in the second direction to be in the measurement area on the basis of the image. In this way, the device 300 may automatically measure the body temperature of an approaching person, and no personal are needed to adjust the orientation of the temperature measuring module 302, which may always face a specific part of a person that needs to be measured.

The control module 303 may further be configured to associate the temperature data measured by the temperature measuring module 302 with the person in response to the positions of the person relative to the temperature measuring module 302 both in the first direction and in the second direction being in the measurement area. In this embodiment, the measurement area in the first direction may be equivalent to the specific distance range described in above embodiments, and the measurement area in the second direction may be equivalent to the measurement area described in above embodiments. When a person is in the measurement area in both the first direction and the second direction, the temperature value measured by the temperature measuring module 302 may be considered the most reliable. Therefore, the control module 303 may associate the measured temperature data with the person or record the temperature data when this condition is met.

In addition to adjusting in the second direction, the adjusting module 305 may further be configured to change the position and/or orientation of the temperature measuring module 302 to adjust the position of the person relative to the temperature measuring module 302 in the first direction. The control module 303 may further be configured to, in response to the position of a person relative to the temperature measuring module 302 in a first direction being in the vicinity of the measurement area, control the adjusting module 305 to adjust the position of the person relative to the temperature measuring module 302 in the first direction to be in the measurement area. For example, when a person walks too close to the device 300 to be measured, the adjusting module 305 may move the temperature measuring module 302 backward for measurement. Otherwise, when the person in the first direction is about to enter the measurement area but stops, the adjusting module 305 may move the temperature measuring module 302 forward for measurement.

In an embodiment, the control module 303 may be implemented by a micro control unit (MCU), which may implement simple control logic. However, due to its limited data processing capabilities, it is impossible to process the visible light images captured by the imaging module 304 by itself. In this case, the control module 303 may transmit the visible light image to an external processing device for processing through the data interface of the device 300. For example, in the above-mentioned device 200, if the second part 222 is equivalent to the device 300, the second part 222 may transmit the visible light image to the processor of the first part 221 for processing through its data interface with the first part 221. In an embodiment, the control module 303 may be implemented by a central processing unit (CPU), which has sufficient processing capability to process visible light images. At this time, the control module 303 may process the visible light image by itself to recognize at least part of a person, or use an external processing device to process the visible light image to recognize at least part of a person, and determine the adjustment direction and the adjustment amount of the adjusting module 305 according to the position of the at least part of a person relative to the measurement area. Therefore, the control module 303 controls the adjusting module 305 on the basis of the visible light image to adjust the position of the person relative to the temperature measuring module 302 in the second direction to be in the measurement area. For example, the control module 303 may determine the direction and angle by which the temperature measuring module 302 needs to be rotated on the basis of the position of the person's face indicated by the image relative to the measurement area, so as to control the temperature measuring module 302 to rotate so that the person's face is in the measurement area, and control the temperature measuring module 302 to measure the temperature of the person's forehead.

The device 300 may perform data transmission with the external through a data interface. In addition to the above-mentioned transmission of visible light images, the control module 303 may also transmit the temperature measured by the temperature measuring module 302 to the external through the data interface. The data interface may be any wired or wireless communication interface, such as a USB interface. The USB interface may not only provide the function of data transmission, but also be used as a power interface. For example, the USB interface may provide a current of 500 mA. If the demand for the current intensity of the device 300 is not higher than this, the USB interface may be used for power supply. In an embodiment, the adjusting module 305 includes a motor for actuation. The motor usually requires a relatively high current intensity, for example, it may be 1000 mA, so the device 300 may further include a power interface to receive a voltage from the external (usually a DC voltage, or an AC voltage). The device 300 may further include a voltage converter, which converts the voltage from the power interface into a corresponding DC voltage to be provided to the imaging module 304, the temperature measuring module 302, the sensing module 301, the control module 303, and the adjusting module 305, respectively. In this way, even if the externally provided voltages have different levels (for example, 12V, 19V, 5V, etc.), the device 300 may work normally.

In an embodiment, the device 300 may be configured to have the appearance structure of the device 100 described above. In another embodiment, the device 300 may be configured to have the appearance structure of the device 200 described above. For example, the imaging module 304, the temperature measuring module 302, and the adjusting module 305 may be configured to be located in the second part 222, the control module 303 may be configured to be in the first part 221, and the sensing module 301 may be configured to be in the first part 221 or support part 210. Thus, the sensing module 301 may sense the distance from an object to the device 300 in lower front of the device 300, i.e. the distance from an object near the ground to the device 300.

In an embodiment, the device 300 may further include a sensor, which may have the function of the sensor as described above. In this embodiment, the sensor may include two sensors, namely a first sensor and a second sensor. The first and second sensors are respectively disposed on opposite sides of the device 300. For example, when the device 300 is configured to have the appearance structure of the device 100, the first and second sensors may be disposed on the left part 111 and the right part 112 of the base 110, respectively. For example, when the device 300 is configured to have the appearance structure of the device 200, the first and second sensors may be respectively disposed on the left and right sides of the second part 222, or may be disposed on the left and right sides (or upper and lower sides) of the first part 221, respectively. The first and second sensors respectively sense, at the first and second positions respectively, whether there is an object within a predetermined range in front of them. When a display prompts the user to operate, the user may use the sensor to operate, so as to avoid contact operation, which is especially suitable for application scenarios with strict requirements on sanitary conditions. For example, the user may place his hand (it will be appreciated that it may also be other parts of the body, or even objects other than the human body) within a predetermined range in front of the first sensor staying for a predetermined time to perform a first operation, such as a confirmation operation. The user may place his hand within a predetermined range in front of the second sensor staying for a predetermined time to perform a second operation, such as a canceling operation. The user may swing his hand from the predetermined range in front of the first sensor into the predetermined range in front of the second sensor (i.e., there is an object in the predetermined range in front of the first sensor, and the object is in the predetermined range in front of the second sensor after a predetermined time period while there is no object in the predetermined range in front of the first sensor) to perform a third operation, such as a left slide operation. The user may also swing his hand from the predetermined range in front of the second sensor into the predetermined range in front of the first sensor (i.e., there is an object in the predetermined range in front of the second sensor, and the object is in the predetermined range in front of the first sensor after a predetermined time period while there is no object in the predetermined range in front of the second sensor) to perform a fourth operation, such as a right slide operation. Both the first and second sensors may be implemented by distance sensors.

FIG. 11 is a block diagram schematically illustrating a device 600 for measuring human body temperature according to an embodiment of the present disclosure. At least a portion of the device 600 may be at least a part of the devices 100, 200, 300 described above. The various functions described above, including the methods, operations, processes, steps, applications, procedures, etc. mentioned above, can be implemented in hardware, software, firmware, or any combination thereof. If implemented in software, the various functions may be implemented by one or more instructions 621 stored on a memory 620, such as a computer readable medium. If implemented in firmware, the various functions may be implemented by the processor 610 executing instructions 621, such as firmware code, stored on the memory 620. If implemented in hardware, various functions can be implemented by processing circuitry.

The at least a portion of the device 600, the control module 303 of the above device 300, for example, includes one or more processors 610 and one or more memories 620, wherein one or more processors 610 are communicably coupled to one or more memories 620. One or more of the one or more memories 620 can be coupled to one or more processors 610 via a bus, port, or network, and/or can be directly coupled or incorporated in any one of the one or more processors 610. Each of the one or more memories 620 can store content that is accessible by one or more processors 610, including instructions 621 that can be executed by one or more processors 610, and data 622 that can be retrieved, manipulated, or stored by one or more processors 610.

The instructions 621 may be any set of instructions to be executed directly, such as machine code, or indirectly, such as scripts, by the one or more processors 610. The instructions 621 may be stored in object code format for direct processing by the one or more processors 610, or in any other computing device language including scripts or collections of independent source code modules that are interpreted on demand or compiled in advance. Functions, methods and routines of the instructions 621 are explained in more detail above.

The one or more memories 620 may be any transitory or non-transitory computer readable storage medium capable of storing contents accessible by the one or more processors 610, such as a hard drive, a memory card, an ROM, an RAM, a DVD, a CD, a USB memory, a write-enabled memory, a read-only memory or the like. The one or more memories 620 may include a distributed storage system where the instructions 621 and/or the data 622 are stored on a plurality of different storage devices which may be physically located at the same or different geographic locations.

The one or more processors 610 may retrieve, store or modify the data 622 in accordance with the instructions 621. The data 622 stored in the one or more memories 620 may include visible light images, thermal radiation images, binary images, measured distances, recognized identities, etc. Those skilled in the art will appreciate that other data may also be stored in one or more memories 620. For example, although the subject matter described herein is not limited by any particular data structure, the data 622 may also be stored in computer registers (not shown) as a table or XML document having many different fields and records stored in a relationship database. The data 622 may also be formatted in any computing device-readable format such as, but not limited to, binary values, ASCII or Unicode. In addition, the data 622 may include any information sufficient to identify relevant information, such as a serial number, descriptive text, a dedicated code, a pointer, references to data stored in other memories such as at other network locations, or information used by a function for computing related data.

The one or more processors 610 may be any conventional processors, such as a commercially available central processing unit (CPU), graphics processing unit (GPU), microprogrammed control unit (MCU), and the like. Alternatively, one or more of the processors 610 may also be dedicated components such as an application specific integrated circuit (ASIC) or other hardware based processor. Although not required, one or more processors 610 may include specialized hardware components to perform particular computing processes, such as checking the frames, etc., faster or more efficiently.

Although one or more processors 610 and one or more memories 620 are shown schematically in the same block in FIG. 11, one or more processors 610 or one or more memories 620 may actually include processors or memories that may exist in the same physical housing or in different physical housings. For example, one of the one or more memories 620 can be a hard drive or other storage medium located in a different housing than the housing of each of the one or more processors 610. Accordingly, references to processor or memory should be understood to include a collection of processors or memories that refer to possible parallel operations or possibly non-parallel operations. Although some of the functions described above are indicated to occur on a single computing device having a single processor, various aspects of the subject matter described herein can be implemented by multiple processors 610, for example, in communication with one another via network.

Moreover, although one or more processors 610 and one or more memories 620 are schematically illustrated in different blocks in FIG. 11, the at least a part of a device 600 can be formed as a component, such as the processors 610, the memories 620, and various peripheral interfaces (such as a USB interface, an A/D conversion interface, and a UART interface, etc.) are integrated into a single chip to form a single chip microcomputer.

It should be noted that the “user” referred to in the present disclosure may be the person being measured, or other persons, such as the supervisor of the temperature measuring device.

Although some specific embodiments of the present disclosure have been described in detail with examples, it should be understood by a person skilled in the art that the above examples are only intended to be illustrative but not to limit the scope of the present disclosure. The embodiments disclosed herein can be combined arbitrarily with each other, without departing from the scope and spirit of the present disclosure. It should be understood by a person skilled in the art that the above embodiments can be modified without departing from the scope and spirit of the present disclosure. The scope of the present disclosure is defined by the attached claims.

What is claimed is:

1. A method for measuring human body temperature, which comprises:
acquiring a distance from an object in front of a temperature measuring device to the temperature measuring device and a visible light image in front of the temperature measuring device;
adjusting an orientation of the temperature measuring device according to the visible light image in response to the distance being less than a threshold, so that a specific part of the person is in a measurement area of the temperature measuring device; and
measuring a temperature of the specific part of the person in response to the specific part of the person being in the measurement area of the temperature measuring device.

2. The method of claim 1, wherein the acquired distance is a distance from an object that is located in lower front of the temperature measuring device to the temperature measuring device, and the method further comprises:
in response to not any part of a person being in the visible light image, adjusting the orientation of the temperature measuring device within its maximum adjustable range and acquiring a visible light image in front of the temperature measuring device at each orientation, until at least part of a person is in the visible light image.

3. The method of claim 1, which further comprises:
associating the temperature of the specific part of the person with the distance.

4. The method of claim 1, which further comprises:
in response to the specific part of the person being in the measurement area of the temperature measuring device, continuously acquiring distances from the person to the temperature measuring device and continuously measuring temperatures of the specific part of the person; and
recording each measured temperature and corresponding distance.

5. The method of claim 1, which further comprises:
prompting the person to be closer to the temperature measuring device in response to the distance from the person to the temperature measuring device being greater than an operable distance.

6. The method of claim 1, which further comprises:
acquiring a thermal radiation image in front of the temperature measuring device in response to not any part of a person being in the visible light image; and
adjusting the orientation of the temperature measuring device according to the thermal radiation image in response to at least part of a person being in the thermal radiation image, so that the specific part of the person is in the measurement area of the temperature measuring device.

7. The method of claim 1, wherein the specific part includes at least one of a face, a forehead, a neck, and a wrist.

8. The method of claim 1, which further comprises:
displaying at least part of the visible light image including the measurement area.

9. The method of claim 1, which further comprises:
recognizing an identity of the person, and associating the temperature measured by the temperature measuring device with the identity of the person.

10. The method of claim 9, which further comprises:
displaying the recognized identity;
receiving a contactless operation from a user, the operation indicating whether the identity is confirmed; and
associating the temperature measured by the temperature measuring device with the identity of the person in response to the identity being confirmed.

11. A device for measuring human body temperature, which comprises a processor, a visible light camera and an infrared thermal imager adjacent to the visible light camera, wherein
the visible light camera is configured to capture a visible light image in its field of view, and the field of view of the visible light camera includes at least part of the field of view of the infrared thermal imager; and
the processor is configured to:
control the infrared thermal imager to capture a first thermal radiation image in its field of view in response to not any part of a person being in the visible light image;
adjust the orientation of the infrared thermal imager according to the first thermal radiation image in response to at least part of a person being in the first thermal radiation image, so that a specific part of the person is in a measurement area of the infrared thermal imager; and
control the infrared thermal imager to capture a second thermal radiation image in its field of view in response to the specific part of the person being in the measurement area of the infrared thermal imager, and measure a temperature of the specific part of the person according to the second thermal radiation image.

12. The device of claim 11, wherein the processor is further configured to:
determine whether pixels of the first thermal radiation image with temperature values greater than a threshold corresponds to at least part of a person according to the temperature value of each pixel of the first thermal radiation image; and
determine that at least part of a person is in the first thermal radiation image in response to the pixels with temperature values greater than the threshold corresponding to at least part of a person.

13. The device of claim 11, wherein the processor is further configured to:
adjust the orientation of the infrared thermal imager according to the visible light image in response to at least part of a person being in the visible light image, so that the specific part of the person is in the measurement area of the infrared thermal imager.

14. The device of claim 11, wherein the device further comprises a distance sensor configured to sense a distance from an object to the device, and the processor is further configured to:
control the infrared thermal imager to capture the second thermal radiation image in its field of view in response to the specific part of the person being in the measurement area of the infrared thermal imager and the distance being less than a threshold.

15. A device for measuring human body temperature, which comprises a sensing module, an adjusting module, a temperature measuring module, and a control module, wherein
the sensing module is configured to sense a position of a person relative to the temperature measuring module in a first direction;
the adjusting module is configured to change a position and/or an orientation of the temperature measuring module so as to at least adjust the position of the person relative to the temperature measuring module in a second direction;
the temperature measuring module is configured to measure a temperature of an object in a measurement area of the temperature measuring module; and
the control module is configured to control the adjusting module to adjust the position of the person relative to the temperature measuring module in the second direction to be in the measurement area, in response to the position of the person relative to the temperature measuring module in the first direction being in the measurement area.

16. The device of claim 15, wherein the control module is further configured to associate the temperature data measured by the temperature measuring module with the person in response to the positions of the person relative to the temperature measuring module both in the first direction and in the second direction being in the measurement area.

17. The device of claim 15, wherein
the adjusting module is further configured to change the position and/or orientation of the temperature measuring module so as to adjust the position of the person relative to the temperature measuring module in the first direction; and
the control module is further configured to control the adjusting module to adjust the position of the person relative to the temperature measuring module in the first direction to be in the measurement area, in response to the position of the person relative to the temperature measuring module in the first direction being in vicinity of the measurement area.

18. The device of claim 15, which further comprises an imaging module, wherein
the imaging module is configured to capture an image of the person; and
the control module is further configured to control the adjusting module to adjust the position of the person relative to the temperature measuring module in the second direction to be in the measurement area based on the image.

19. The device of claim 15, wherein the person includes a specific part of the person.

20. The device of claim 15, wherein the first direction is a direction extending forwardly from the temperature measuring module, and the second direction is a direction in a plane substantially perpendicular to the first direction.
